(19) United States
(12) Patent Application Publication
(10) Pub. No.: US 2016/0081603 A1
(43) Pub. Date: Mar. 24, 2016

(54) Reflection-Type Multi-Sensor Array Blood Oxygen Detection Device

(71) Applicants: Wei PENG, Hubei (CN); WUHAN YUANGUANG RUIKANG TECHNOLOGY CO., LTD., Hubei (CN)

(72) Inventors: Xiaochang Liu, Wuhan (CN); Qiang Peng, Wuhan (CN)

(21) Appl. No.: 14/891,457

(22) PCT Filed: Apr. 16, 2014
(86) PCT No.: PCT/CN2014/075439

§ 371 Date: Nov. 17, 2015

(30) Foreign Application Priority Data
May 17, 2013 (CN) 201320272422.5

(51) Int. Cl. A61B 5/1455; A61B 5/00

(52) U.S. Cl. CPC A61B 5/14552; A61B 5/6831; A61B 2562/04

(57) ABSTRACT

The utility model discloses a reflection-type multi-sensor array blood oxygen detection device, which comprises an apparatus body, a photosensitive sensor array, a light transmitter array and a bandage, wherein the light transmitter array is distributed around the photosensitive sensor array; and a light shielding plate is arranged between the light transmitter array and the photosensitive sensor array. The blood oxygen detection device can be used for effectively measuring the degree of blood oxygen saturation of the human issue by detecting the reflected light of the human tissue.

### TECHNICAL FIELD

The utility model belongs to the technical field of medical apparatus, and in particular relates to a reflection-type multi-sensor array blood oxygen detection device.

### BACKGROUND

With the accelerated pace of modern life and the weighted pressure of people's life, SDB (sleep-disordered breathing) and subsequent problems have become a prominent medical & public health issue and have drawn attention of people. According to international authoritative journals' classification on sleep diseases, more than 90 sleep diseases are caused by external or internal factors, and the prevalent rate of such most common diseases as insomnia and SDB is quite high in China. In the preliminary screening of SDB, it generally involves the acquisition of blood oxygen pulse parameters; most oximeters for the preliminary screening of SDB on the market adopt a single transmitting & receiving sensor for transmission-type detection; according to the method, a luminescent tube used as a transmitter and a photosensitive sensor serving as a receiver are located on two sides of detected human tissue; red light and infrared light transmitted from the transmitter penetrate the human tissue, and the photosensitive sensor is used for receiving the transmission light; since hemoglobin and oxyhaemoglobin in the blood are different in absorption rate on light, the transmission light acquired by the photosensitive sensor has different data; the data is transmitted to an apparatus body having data processing function and display function and is processed by the apparatus body, so that the degree of blood oxygen saturation of a human body to be detected can be displayed by the apparatus body. During using the transmission-type oximeter, a finger cover shall be worn generally, which is easy to cause discomfort on the finger in long-term measurement and is difficult to guarantee the measurement accuracy for patients relatively poor in finger-tip circulation. In recent years, some scholars also propose the way to effectively measure the degree of blood oxygen saturation of human tissue by detecting the reflected light of the hemoglobin and oxyhaemoglobin of the human tissue instead of the transmission light energy. But the market is in lack of a reflection-type blood oxygen detection apparatus which is good in effect.

### SUMMARY OF THE INVENTION

The utility model aims at providing a reflection-type multi-sensor array blood oxygen detection device, wherein the blood oxygen detection device can be used for effectively measuring the degree of blood oxygen saturation of the human issue by detecting the reflected light of the human tissue.

In order to achieve the purpose, the reflection-type multi-sensor array blood oxygen detection device of the utility model comprises an apparatus body, a photosensitive sensor array, a light transmitter array and a bandage; the light transmitter array is distributed around the photosensitive sensor array; and a light shielding plate is arranged between the light transmitter array and the photosensitive sensor array.

Preferably, the light shielding plate is additionally arranged at the top of the photosensitive sensor array and just the inner side part close to the skin is reserved to receive the reflected light, so that the light transmitted from luminescent device fails to irradiate on the photosensitive sensor, and the sensitivity and the accuracy of the photosensitive sensor are improved.

Furthermore, the photosensitive sensor array is of a round, square or polygonal structure; corresponding, the light transmitter array is of a round, hollow square or polygonal structure matched with the photosensitive sensor array, and is arranged around the photosensitive sensor array.

According to the reflection-type multi-sensor array blood oxygen detection device disclosed by the utility model, the light transmitter array is distributed around the photosensitive sensor array; during detecting, both the light transmitter array and the photosensitive sensor array are located on the same side of human tissue to be detected; light beams of the hemoglobin and oxyhaemoglobin in the human tissue to the light transmitter array are reflected and are received by the photosensitive sensor in the middle; the reflected light data acquired by the photosensitive sensor is transmitted to an apparatus body having data processing function and display function and is processed by the apparatus body, so that a purpose of measuring the degree of blood oxygen saturation by detecting the reflected light of the human tissue is achieved. Meanwhile, since the light transmitter array and the photosensitive sensor array are located on the same side of the human tissue to be detected, it can measure on any position of the surface artery without a finger cover, such as the parts of wrist radial artery, carotid artery and the like. When conducting preliminary screening on the SDB, the detection device disclosed by the utility model can be used for relieving the discomfort such as finger-tip paralysis caused by long-term wearing of the finger cover and can be used for avoiding the influence on the measurement quality and even the interrupt of the acquired signal caused by relative motion between measurement equipment and body with the motion of the body.

On the basis of sensor array technology, illumination intensity and the intensity of the received signal are greatly improved compared with a single sensor, so that the stability and the reliability of the reflection-type blood oxygen detection device are integrally improved. By a way of arranging the luminescent device around the photosensitive sensor array, the illumination and the receiving area are increased; and the detection device disclosed by the utility model can be used for measuring without precisely aligning the sensor to the artery, so that the usability of the equipment is enhanced.

### BRIEF DESCRIPTION OF THE DRAWINGS

FIG. 1 is a structural schematic diagram of the reflection-type multi-sensor array blood oxygen detection device.

FIG. 2 is a vertical structural schematic diagram of the reflection-type multi-sensor array blood oxygen detection device as shown in FIG. 1.

FIG. 3 is a schematic diagram of the detection light line of the reflection-type multi-sensor array blood oxygen detection device during working.

### DESCRIPTION OF THE EMBODIMENTS

The reflection-type multi-sensor array blood oxygen detection device of the utility model will be further illustrated in details in conjunction with attached drawings and specific embodiments:

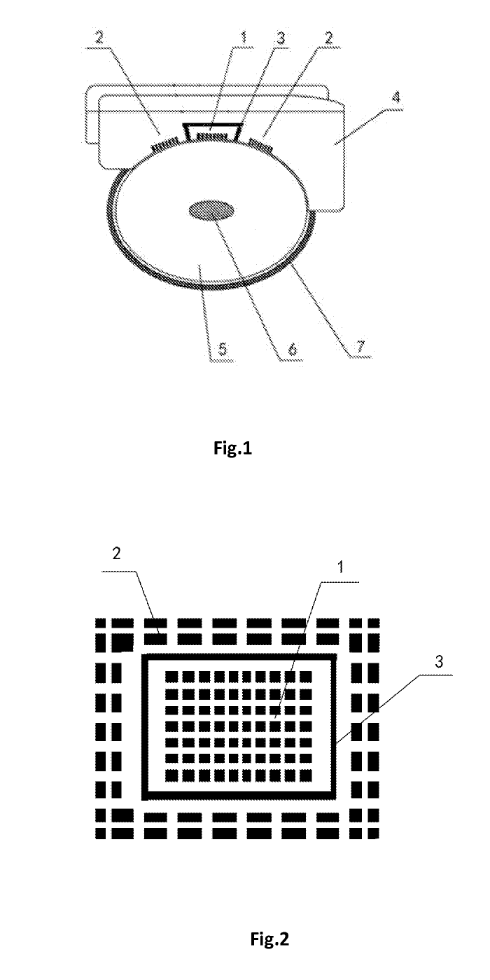

As shown in the FIG. 1, the reflection-type multi-sensor array blood oxygen detection device comprises an apparatus body 4, a photosensitive sensor array 1, a light transmitter array 2 and a bandage 7, wherein the light transmitter array 2 is distributed around the photosensitive sensor array 1; a light shielding plate 3 is arranged between the light transmitter array 2 and the photosensitive sensor array 1; the light shielding plate 3 is located around and at the top of the photosensitive sensor array 1 and just the inner side part close to the skin is reserved to receive the reflected light; the photosensitive sensor array 1 is of a square structure; and correspondingly, the light transmitter array 2 is of a hollow square structure matched with the photosensitive sensor array 1 and is arranged around the photosensitive sensor array 1 (as shown in FIG. 2).

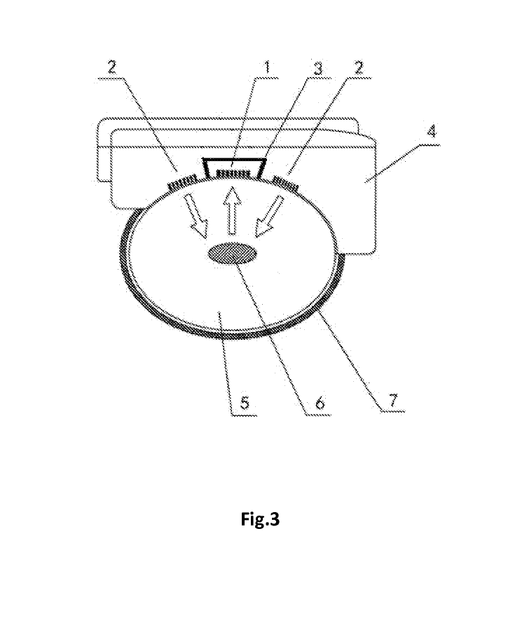

When the reflection-type multi-sensor array blood oxygen detection device disclosed by the utility model is used for measuring degree of blood oxygen saturation of human body, the bandage 7 is wound on the detected part 5 of the human body. During detecting, as shown in FIG. 3, red light and infrared light transmitted by the light transmitter array 2 are reflected by blood 6 in the human tissue and are received by the photosensitive sensor array 1; the reflected light data acquired by the photosensitive sensor is transmitted to an apparatus body 4 having data processing function and display function and is processed by the apparatus body, so that the degree of blood oxygen saturation of the human body to be detected can be displayed.

1. A reflection-type multi-sensor array blood oxygen detection device, which comprises an apparatus body (4), wherein, the reflection-type multi-sensor array blood oxygen detection device further comprises a photosensitive sensor array (1), a light transmitter array (2) and a bandage (7), wherein the light transmitter array (2) is distributed around the photosensitive sensor array (1); and a light shielding plate (3) is arranged between the light transmitter array (2) and the photosensitive sensor array (1).

2. The reflection-type multi-sensor array blood oxygen detection device according to claim 1, wherein, the light shielding plate (3) is additionally arranged at the top of the photosensitive sensor array (1).

3. The reflection-type multi-sensor array blood oxygen detection device according to claim 1, wherein, the photosensitive sensor array (1) is of a round, square or polygonal structure; and the light transmitter array (2) is of a round, hollow square or polygonal structure matched with the photosensitive sensor array (1).

4. The reflection-type multi-sensor array blood oxygen detection device according to claim 2, wherein, the photosensitive sensor array (1) is of a round, square or polygonal structure; and the light transmitter array (2) is of a round, hollow square or polygonal structure matched with the photosensitive sensor array (1).
